(19) United States
(12) Patent Application Publication
(10) Pub. No.: US 2022/0194897 A1
(43) Pub. Date: Jun. 23, 2022

(54) NOVEL ENOL-ACETATES

(71) Applicant: DSM IP ASSETS B.V., Heerlen (NL)

(72) Inventors: Werner BONRATH, Kaiseraugst (CH); Marc-André MUELLER, Kaiseraugst (CH); Bettina WUESTENBERG, Kaiseraugst (CH); Florian ZIEGLER, Kaiseraugst (CH)

(21) Appl. No.: 17/603,122

(22) PCT Filed: Apr. 3, 2020
(86) PCT No.: PCT/EP2020/059483

§ 371 Date: Oct. 12, 2021

(30) Foreign Application Priority Data
Apr. 15, 2019 (EP) 19169207.8

(51) Int. Cl. C07C 403/12

(52) U.S. Cl. CPC C07C 403/12

(57) ABSTRACT

The present invention relates to new specific enol esters of formula (I) as well as to a process for their production. In formula (I) R is COR′ where R′ is a C₁-C₁₆ alkyl group.

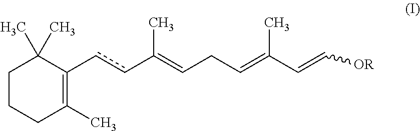

The present invention relates to new specific enol acetates as well as to their production.

Enol acetates are important intermediates in various organic syntheses.

The new enol acetates we have found are those of formula (I)

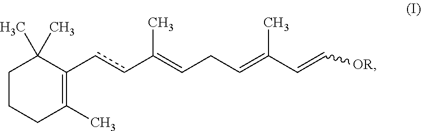

wherein
R is —COR′, wherein R′ is a C₁-C₁₆ alkyl group.

The dotted line signifies an optional double bond. This means that the compound of formula (I) represents the following two compounds of formula (I′) and (I″)

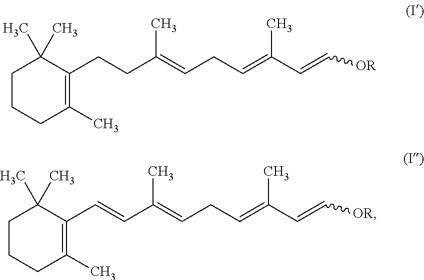

wherein
R is —COR′, wherein R′ is a C₁-C₁₆ alkyl group.

There are two isomers (compound of formula (Ia) and (Ib))

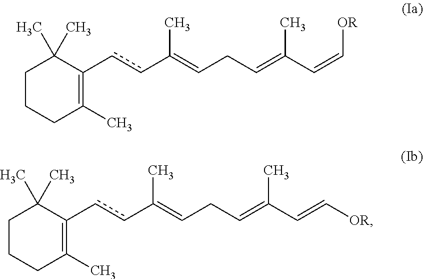

wherein
R is —COR′, wherein R′ is a C₁-C₁₆ alkyl group.

Each of these isomers can have an additional double bond, which is represented by the dotted line. Due to the C—C-double bonds, there is variety of stereoisomeric forms. This means that the formula of compound (Ia) and (Ib) represent the following compounds of formula (Ia′), (Ia″), (Ib′) and (Ib″):

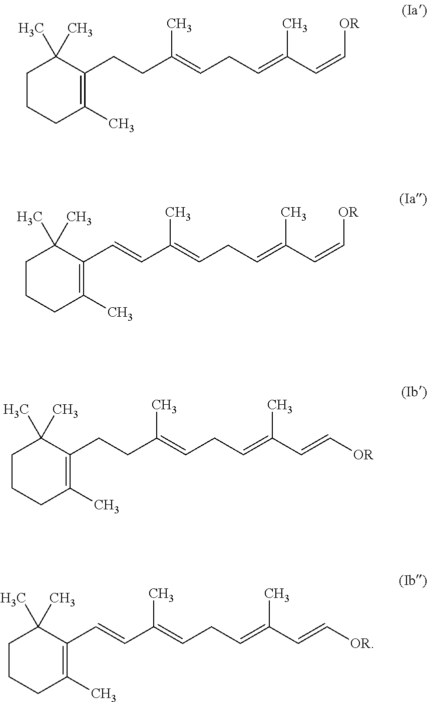

Therefore, the present invention relates to compounds of formula (I)

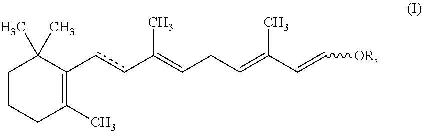

wherein
R is —COR′, wherein R′ is a C₁-C₁₆ alkyl group (preferably a C₁, C₂ or C₁₅-alkyl group).

Therefore, the present invention relates to the compounds of formula (Ia)

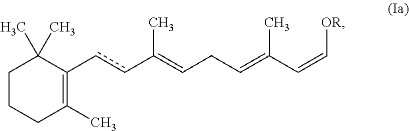

wherein
R is —COR′, wherein R′ is a C₁-C₁₆ alkyl group (preferably a C₁, C₂ or C₁₅-alkyl group).

Therefore, the present invention relates to the compounds of formula (Ib)

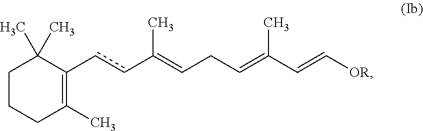

wherein
R is —COR′, wherein R′ is a C₁-C₁₆ alkyl group (preferably a C₁, C₂ or C₁₅-alkyl group).

Therefore, the present invention relates to the compounds of formula (Ia′)

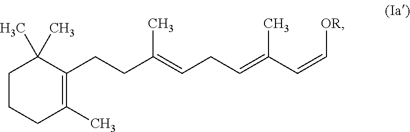

wherein
R is —COR′, wherein R′ is a C₁-C₁₆ alkyl group (preferably a C₁, C₂ or C₁₅-alkyl group).

Therefore, the present invention related to the compounds of formula (Ia″)

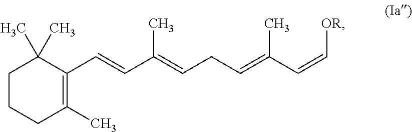

wherein
R is —COR′, wherein R′ is a C₁-C₁₆ alkyl group (preferably a C₁, C₂ or C₁₅-alkyl group).

Therefore, the present invention relates to the compounds of formula (Ib′)

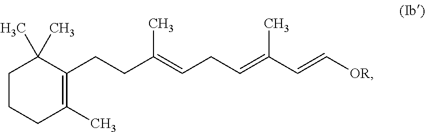

wherein
R is —COR′, wherein R′ is a C₁-C₁₆ alkyl group (preferably a C₁, C₂ or C₁₅-alkyl group).

Therefore, the present invention related to the compounds of formula (Ib″)

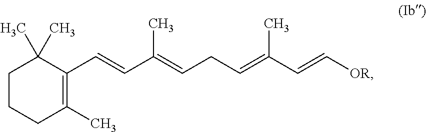

wherein
R is —COR′, wherein R′ is a C₁-C₁₆ alkyl group (preferably a C₁, C₂ or C₁₅-alkyl group).

These new enol acetates are important and useful intermediates in organic syntheses (especially in the synthesis of vitamin A and/or its derivatives).

The following reaction schemes shows how these intermediates (Ia) and (Ib) are used to obtain Vitamin A (and/or its derivatives):

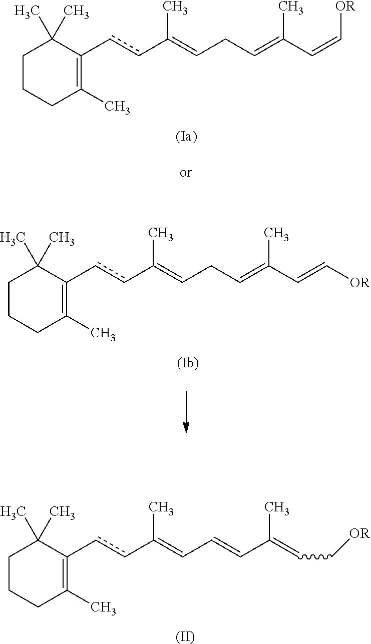

vitamin A and derivatives
   wherein R has the same meanings as defined above.

The enol acetate according to the present invention are produced by an enol-acetate formation of the compounds of formula (III)

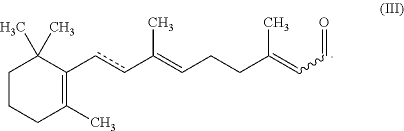

Compounds of formula (III) have two isomers of the following formula (IIIa) and (IIIb):

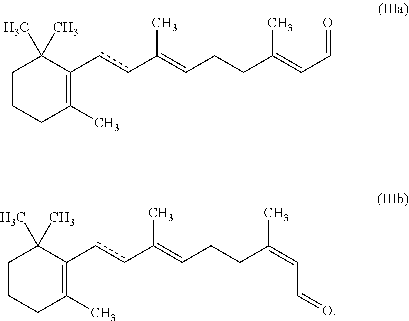

The process is carried out in the presence of at least one acetylating agent, which is a compound of formula (IV)

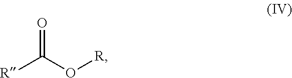

wherein

### R is —COR′ or

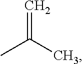

wherein R′ is a C₁-C₁₆ alkyl group (preferably a C₁, C₂ or C₁₅-alkyl group)
R″ is a C₁-C₁₆ alkyl group (preferably a C₁, C₂ or C₁₅-alkyl group).

Alternatively, the process of the present invention can be carried out in the presence of a transition metal catalyst. Especially in the presence of a Cu catalyst. Especially a Cu(III) catalyst. Very suitable is Cu(Ac)₂ as a catalyst.

Due to the C—C double bonds, the compounds of formula (I) as well as the compounds of formula (II) can have several stereochemical isomers, which are not all implicitly drawn in this application, but which are also covered by the present invention.

Therefore, the present invention relates to a process (P) for the production of the compounds of formula (I)

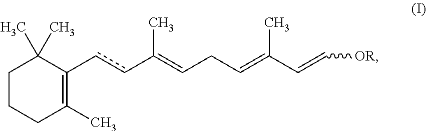

wherein
R is —COR′, wherein R′ is a C₁-C₁₆ alkyl group (preferably a C₁, C₂ or C₁₅-alkyl group)
by acetylation of compounds of formula (III)

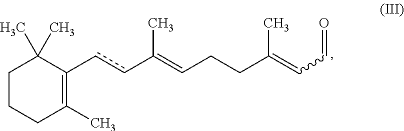

by using at least one acetylating agent of formula (IV)

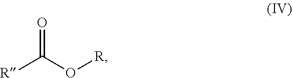

wherein

### R is —COR′ or

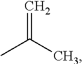

wherein R′ is a C₁-C₁₆ alkyl group (preferably a C₁, C₂ or C₁₅-alkyl group)
R″ is a C₁-C₁₆ alkyl group (preferably a C₁, C₂ or C₁₅-alkyl group).

Alternatively (optionally), the process according to the present invention can be carried out in the presence of at least one transition metal catalyst; especially in the presence of a Cu catalyst. Especially a Cu(II) catalyst. Very suitable is Cu(Ac)₂ as a catalyst.

The amount of the catalyst used in the process according to the present invention can vary. The amount of the catalyst usually goes from 0.001 mol-equivalent up to 0.01 mol-equivalent (in relation to compound of formula (II)).

The process according to the present invention is usually carried out in the presence of at least one organic acid or in the presence of a base. Especially in the presence of p-toluenesulfonic acid.

The amount of the acid or of the base can vary. It goes usually from 0.005 mol-equivalent up to 0.1 mol-equivalent (in relation to compound of formula (II)).

The reaction can be carried out in an inert solvent or the reaction can be carried out without a solvent. Preferably no solvent is used.

The process according to the present is usually carried out at elevated temperatures. Usually the process according to the present invention is carried out at a temperature of from 0° C.-100° C., preferably from 5° C.-90° C.

As stated above the process according to the present invention is one important step in the synthesis of vitamin A (and/or its derivatives).

The following examples serve to illustrate the invention. The temperature is given in ° C. and all percentages are related to the weight.

### EXAMPLES

### Example 1

A flame-dried 2-necked flask equipped with a reflux condenser was charged with p-toulenesulfonic acid (dry, 0.01 eq), hydroquinone (0.01 eq), copper(II)acetate (0.004 eq), isopropenylacetate (2.0 eq) and 3,7-dimethyl-9-(2,6,6-trimethylcyclohex-1-en-1-yl)nona-2,6-dienal (1.0 eq) in the given order. The reaction mixture was stirred for 3 h at 60° C., cooled to room temperature and CH₂Cl₂ (50 mL) was added. The solution was washed with aqueous sat. NaHCO₃-solution (30 mL). The aqueous phase was extracted with CH₂Cl₂ (30 mL) and the combined organic layers were concentrated under reduced pressure (45° C., 2 mbar). The crude material was purified by column chromatography (Heptane, CH₂Cl₂) to afford the product as mixture of isomers.

### Example 2

A flame-dried 2-necked flask equipped with a reflux condenser was charged with p-toulenesulfonic acid (dry, 0.01 eq), hydroquinone (0.01 eq), copper(II)acetate (0.004 eq), isopropenylacetate (2.0 eq) and 3,7-dimethyl-9-(2,6,6-trimethylcyclohex-1-en-1-yl)nona-2,4,6-trienal (1.0 eq) in the given order. The reaction mixture was stirred for 3 h at 60° C., cooled to room temperature and Et₂O (10 mL) was added. The solution was washed with aqueous sat. NaHCO₃-solution (5 mL). The aqueous phase was extracted with Et₂O (5 mL) and the combined organic layers were concentrated under reduced pressure. Crude material was purified by digestion in acetonitrile to afford the product as mixture of isomers.

1. Compounds of formula (I) 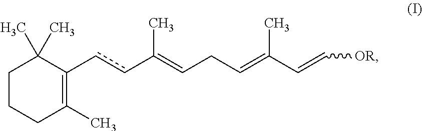
wherein
R is —COR′,
wherein R′ is a C₁-C₁₆ alkyl group (preferably a C₁, C₂ or C₁₅-alkyl group).

2. Compounds according to claim 1, which has formula (Ia) 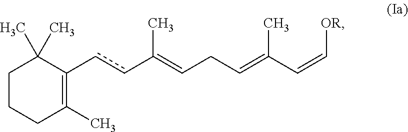
wherein
R is —COR′, wherein R′ is a C₁-C₁₆ alkyl group (preferably a C₁, C₂ or C₁₅-alkyl group).

3. Compounds according to claim 1, which has formula (Ib) 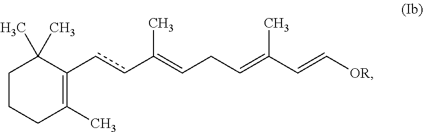
wherein
R is —COR′,
wherein R′ is a C₁-C₁₆ alkyl group (preferably a C₁, C₂ or C₁₅-alkyl group).

4. Process for the production of the compounds of formula (I) according to claim 1 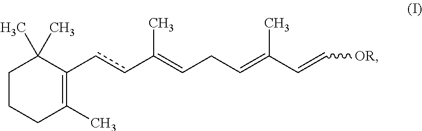
wherein
R is —COR′,
wherein R′ is a C₁-C₁₆ alkyl group (preferably a C₁, C₂ or C₁₅-alkyl group).
by using at least one acetylating agent of formula (IV) 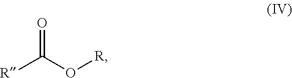
wherein
R is —COR′ or 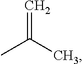
wherein R′ is a C₁-C₁₆ alkyl group (preferably a C₁, C₂ or C₁₅-alkyl group) and
R″ is a C₁-C₁₆ alkyl group (preferably a C₁, C₂ or C₁₅-alkyl group).

5. Process according to claim 4, wherein the process is carried out in the presence of at least one transition metal catalyst.

6. Process according to claim 4, wherein the amount of the catalyst is 0.001 mol-equivalent up to 0.01 mol-equivalent (in relation to compound of formula (II)).

7. Process according to claim 4, wherein the process is carried out in the presence of at least one organic acid.

8. Process according to claim 4, wherein the process is carried out in the presence of at least one base.

9. Process according to claim 7, wherein the amount of the acid or base is 0.005 mol-equivalent up to 0.1 mol-equivalent (in relation to compound of formula (II)).

10. Process according to claim 4, wherein the process is carried out in an inert solvent.

11. Process according to claim 4, wherein the process is carried out without any solvent.

12. Process according to claim 4, wherein the process is carried out at a temperature of from 0° C.-100° C. (preferably from 5° C.-90° C.).
